(19) United States
(12) Patent Application Publication
(10) Pub. No.: US 2012/0109145 A1
(43) Pub. Date: May 3, 2012

(54) CIRCUMCISION DEVICE

(75) Inventor: Emily Gumkowski, Durham, CT (US)

(73) Assignee: Tyco Healthcare Group LP

(21) Appl. No.: 13/192,640

(22) Filed: Jul. 28, 2011

Related U.S. Application Data
(60) Provisional application No. 61/409,581, filed on Nov. 3, 2010.

(51) Int. Cl. A61B 17/326

(52) U.S. Cl. 606/118

(57) ABSTRACT

A circumcision device for removing excess foreskin from a penis is disclosed and includes a crown and a clamp. The crown is oval in shape and defines a proximal end, a distal end, and a first series of guides extending proximally along the proximal end. Any two of the first series of guides define a gap between the two first guides. Each of the first series of guides is equally spaced about a circumference of the oval. The clamp has a first half section and a second half section. The first and second half sections are jointed together with a coupling mechanism. Each of the first half section and the second half section defines a proximal end, a distal end, a free end, a fixed end, and an arc between the free and fixed ends. A second series of guides protrude proximally along the proximal end of each leg. Any two of the second series of guides define a gap between the two second guides.

### CROSS REFERENCE TO RELATED APPLICATION

The present application claims the benefit of and priority to U.S. Provisional Application Ser. No. 61/409,581 filed on Nov. 3, 2010, the entire contents of which are incorporated herein by reference.

### BACKGROUND

1. Technical Field

This application generally relates to the field of circumcision. More particularly, the present disclosure relates to a method and a device for surgical removal of the excess foreskin or prepuce from the penis.

2. Related Art

The conventional circumcision is usually performed by a surgical team consisting of a surgeon and at least one assistant. The team severs the excessive foreskin, which covers glans of the penis, and sutures the cut edge of the foreskin. The average operating time that a conventional circumcision operation consumes is approximately half an hour, a time period considered relatively long for an operation.

The conventional circumcision clamp has another disadvantage in that it causes severe bleeding during the surgery. Generally, most patients experience apprehension during a circumcision operation. During the operation, the patient's penis is almost in a naturally flaccid state.

### SUMMARY

The present disclosure is directed to a circumcision device for surgically removing excess foreskin or prepuce from a penis. The circumcision device includes a crown and a clamp. The crown is oval in shape, and defines a proximal end, a distal end, and a first series of guides extending proximally along the proximal end. Any two of the first series of guides define a gap between the two first guides. Each of the first series of guides may be equally spaced about a circumference of the oval.

The clamp has a first half section and a second half section. The first and second half sections are jointed together with a coupling mechanism. Each of the first half section and the second half section defines a proximal end, a distal end, a free end, a fixed end and an arc between the free and fixed ends. A second series of guides protrude proximally along the proximal end of each leg. Any two of the second series of guides define a gap between the two second guides.

The crown and the clamp may be constructed from a biocompatible material.

The coupling mechanism includes a first boss or pin portion, a second boss or an eyelet portion, and a cap. The first boss extends radially outward from the first half section and includes a distal protrusion. The second boss extends radially outward from the second half section and defines an aperture therethrough. The cap has a head and a securing protrusion. The securing protrusion defines a central recess. The recess is sized larger than the distal protrusion of the pin portion. The securing protrusion is sized smaller than an internal diameter of the aperture of the eyelet portion.

The clamp also has a latching mechanism to secure the free ends of the first and second half sections in a closed position.

The coupling mechanism removably attaches the clamp about the crown. The second series of guides of the clamp overlap about the first set of guides of the crown when the clamp is properly located about the crown.

A method of use of the circumcision device is also provided. The surgeon is provided with the circumcision device. The crown is placed on the glans penis. The foreskin is pulled over the crown. The foreskin is held in place over the crown and the clamp is located over the foreskin and about the crown, such that each of the second series of guides is aligned with a guide from the first series of guides and each of the second gaps is aligned with one of the first gaps. At this time, the clamp is secured about both the foreskin and the crown. Upon completion of properly securing the clamp, the captured foreskin is removed by cutting the foreskin with a scalpel along a proximal edge of the guides. A suture is then inserted and tied in the foreskin extending from each of the first and second gaps. The clamp is then removed and the crown is removed.

### BRIEF DESCRIPTION OF THE DRAWINGS

The accompanying drawings, which are incorporated in and form part of the specification, illustrate the present disclosure when viewed with reference to the description, wherein:

FIG. 1 is a perspective view of a circumcision device in accordance with the principles of the present disclosure, illustrating a clamp fastened about a crown;

FIG. 2 is another perspective view of the device of FIG. 1, illustrating the clamp fastened about the crown;

FIG. 3 is front view of the device of FIGS. 1 and 2;

FIG. 4 is top plan view of the device of FIGS. 1-3;

FIG. 5 is right hand side view of the device of FIGS. 1-4;

FIG. 6 is a perspective view of the crown in accordance with the principles of the present disclosure;

FIG. 7 is another perspective view of the crown in accordance with the principles of the present disclosure;

FIG. 8 is a perspective view of the clamp in an open position, illustrating a pair of legs in a first open position;

FIG. 9 is an exploded perspective view, with parts separated, of the clamp of FIG. 1-5;

FIG. 10 is a side view of the crown placed over the penis glans;

FIG. 11 is a side view of the clamp being placed about the prepuce of the penis and the crown with the crown placed over the penis glans;

FIG. 12 is a perspective view of the clamp placed about the prepuce of the penis and the crown with the crown placed over the penis glans; and

FIG. 13 is a perspective view of the clamp placed about the prepuce of the penis and the crown with the crown placed over the penis glans and the prepuce separated from the penis at a location along the proximal end of the crown and clamp and with sutures secured between the guides.

Other features of the present disclosure will become apparent from the following detailed description, taken in conjunction with the accompanying drawings, which illustrate, by way of example, the principles of the present disclosure.

### DETAILED DESCRIPTION OF THE EMBODIMENTS

Embodiments of the presently disclosed circumcision device and a method for use are described in detail with reference to the drawings, in which like reference numerals designate identical or corresponding elements in each of the several views. As used herein, the term “distal” refers to that portion of the circumcision device, or component thereof, farther from the user while the term “proximal” refers to that portion of the circumcision device or component thereof, closer to the user.

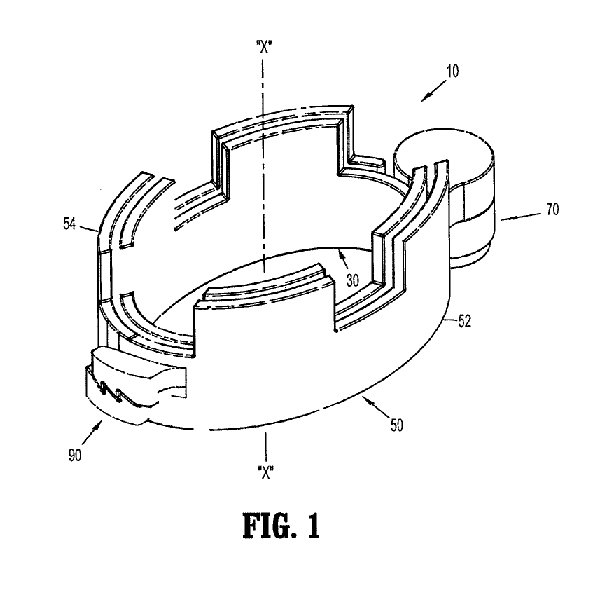

Referring now to the drawings, wherein like reference numerals identify identical or similar structural elements of the subject device throughout the several views, there is illustrated in FIG. 1 a circumcision device, designated generally by reference numeral 10.

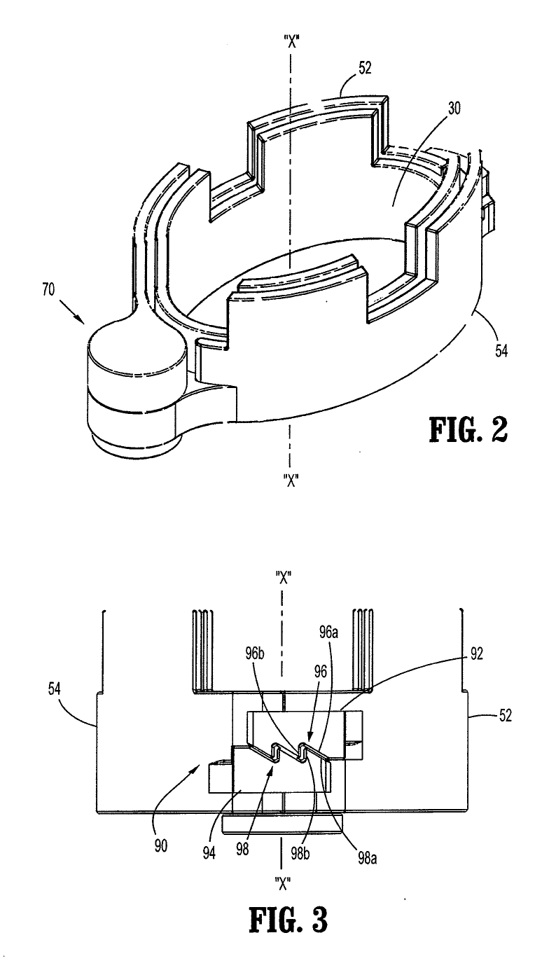

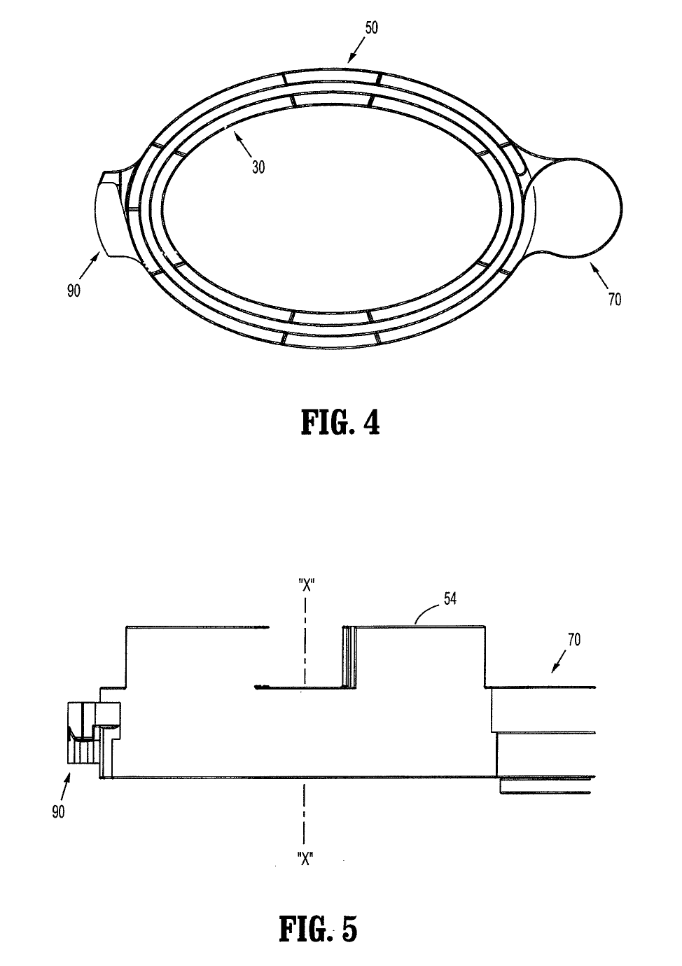

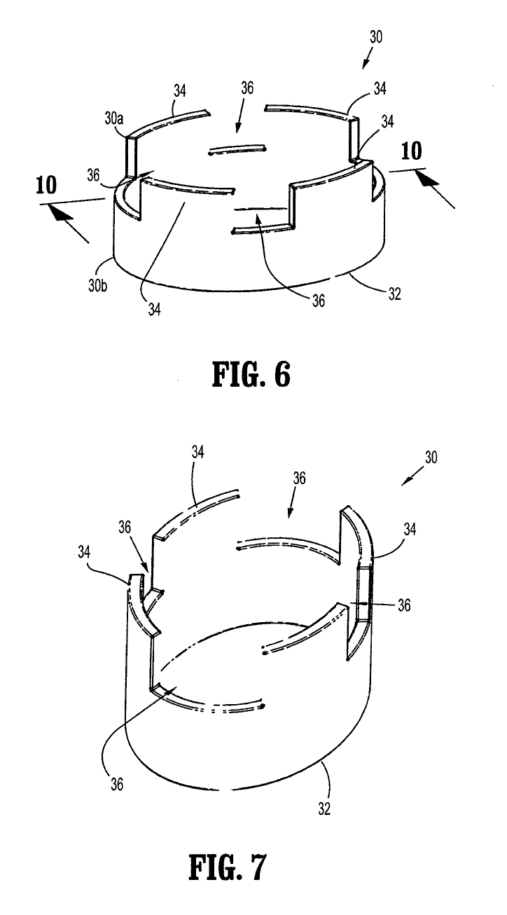

With reference to FIGS. 1-5, the circumcision device 10 includes a crown 30 and a clamp 50. With reference to FIGS. 6 and 7, the crown 30 is generally oval in shape and is sized to fit into the clamp 50. A longitudinal axis ‘X’ is defined through the center of the oval. The crown 30 defines a proximal end 30a and a distal end 30b.

The crown 30 includes a base section 32 and a first series of guides 34. The base section 32 is continuous about the distal end 30b and extends proximally therefrom. Each of the first series of guides 34 extend proximally from the base section 32 to the proximal end 30a. Any two adjacent guides of the first series of guides define a gap 36 therebetween. Each gap 36 extends the height of the first series of guides 34. Each of the first series of guides 34 may be equally spaced about a circumference of the oval.

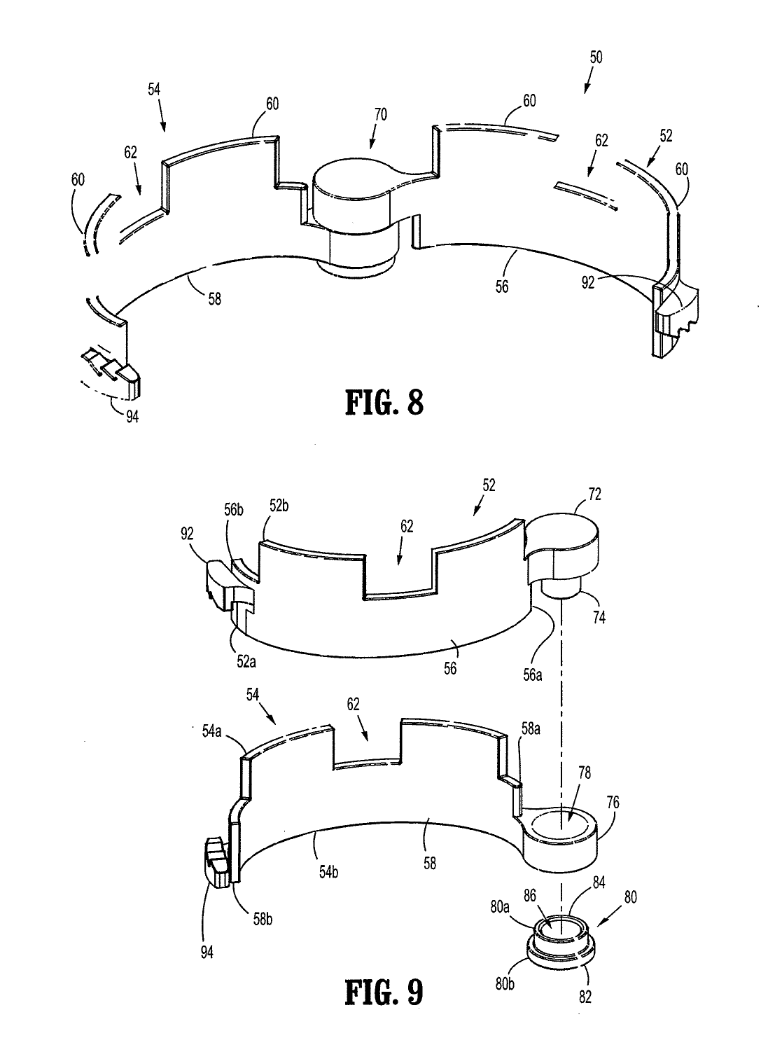

With reference to FIGS. 8 and 9, the clamp 50 has a first half section 52 and a second half section 54. The first half section 52 and the second half section 54 are pivotally connected to each other by a coupling mechanism 70. Each of the first half section 52 and the second half section 54 defines a proximal end 52a, 54a and a distal end 52b, 54b. The first half section 52 has a first leg 56 that extends along the distal end 52b. The second half section 54 has a second leg 58 that extends along the distal end 54b. Both the first leg 56 and the second leg 58 are arcuate in shape from a first fixed or joined end 56a, 58a to a second free end 56b, 58b. A second series of guides 60 extend proximally from each of the first leg 56 and second leg 58.

Any two adjacent guides of the second series of guides 60 define a gap 62 therebetween. Each of the gaps 62 extends the height of the second series of guides 34. Each of the second series of guides 60 may be equally spaced along the first half section 52 and second half section 54.

The crown 30 and the clamp 50 may be constructed from a biocompatible material.

The coupling mechanism 70 includes a pin portion 72 extending from the first half section 52, and an eyelet portion 76 extending from the second half section 54, and a cap 80. The pin portion 72 has a hinge pin 74 extending distally therefrom. The eyelet portion 76 defines a hinge aperture 78 extending therethrough.

The cap 80 has a head 82 and a securing protrusion 84 extending proximally from the head 82. The cap 80 defines a proximal end 80a, a distal end 80b, and a central recess 86 extending distally from the proximal end 80a of the securing protrusion 84. The central recess 86 defines an internal diameter that is substantially equal to an external diameter of the hinge pin 74. The central recess 86 and the hinge pin 74 are sized to cause an interference fit or a snap fit between inner walls of the central recess 86 and the hinge pin 74. The securing protrusion 84 defines an outer diameter that is slightly smaller than the hinge aperture 78 to allow the securing protrusion 84 of the cap 80 to fit into the hinge aperture 78 of eyelet portion 76.

With reference to FIG. 3, the clamp 50 includes a latching mechanism 90. The latching mechanism 90 includes a first arm 92 extending from the free end 56b of the first half section 52 and a second arm 94 extending from the free end 58b of the second half section 54. The first arm 92 and the second arm 94 are offset, so that when the free ends 56b, 58b are brought together, the first arm 92 and the second arm 94 overlap. Both the first arm 92 and the second arm 94 include a set of teeth 96, 98 that extend toward each other. The first set of teeth 96 extend distally from the first arm 92 and the second set of teeth 98 extend proximally from the second arm 94. Each set of teeth have an angled surface 96a, 98a that is angled with respect to the longitudinal axis ‘X’ and a vertical surface 96b, 98b that is substantially parallel to the longitudinal axis ‘X’. The angled surfaces 96a, 98a allow the arms 92, 94 to slide together and the parallel surfaces 96b, 98b prevent the arms 92, 94 from sliding apart or unintentionally separating.

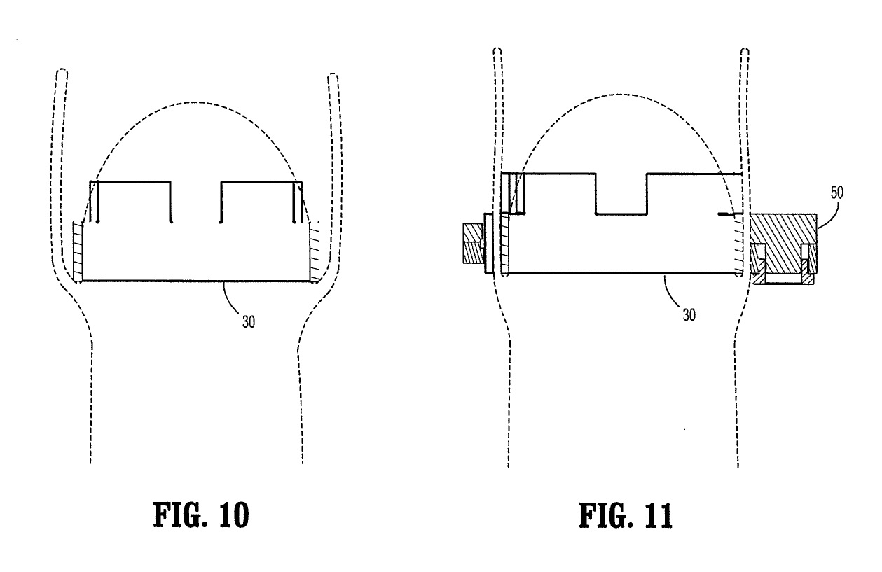

With reference to FIGS. 10-13, a method of use of the circumcision device to circumcise a penis to remove an excess of foreskin or prepuce from the penis is also provided. The surgeon is provided with the circumcision device 10. Referring now to FIG. 10, the crown 30 is placed inside of the foreskin and about the glans penis. The excess of prepuce is fully extended or pulled over the crown 30 until the desired tension of prepuce is reached. This ensures that there is no bunching of the foreskin about the crown 30.

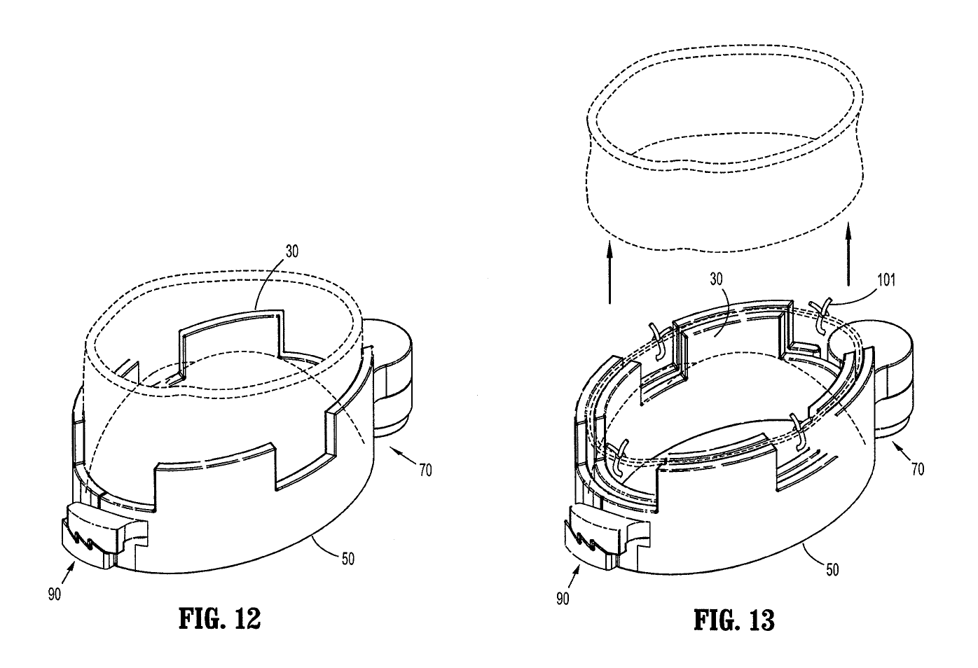

Referring now to FIGS. 11 and 12, While the foreskin is held in the lengthened position, the clamp 50 is placed over the foreskin and about the internally placed crown 30.

Referring now to FIGS. 1 and 13, the oval shape of the crown 30 provides a self-aligning feature with the clamp 50. As a result, when the clamp 50 is placed about the crown 30, the second series of guides 60 of the clamp 50 align with the first series of guides 34 of the crown 30. Therefore, the gaps 36 in the crown 30 also align with the gaps 62 in the clamp 50.

The clamp 50 is then secured in place about the foreskin and crown 30 by the latching mechanism 90. The proximal ends 52a, 54a of the second series of guides 60 are used as a template for directing a scalpel or shearing instrument (not shown for clarity) in the removal of the foreskin by a cutting action.

A suture 101 is then inserted in the foreskin extending from each of the gaps 36, 62. The suture 101 is then tied and the clamp 50 and crown 30 are removed.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A circumcision device for removing tissue from a penis, comprising:
a crown being oval in shape, the crown defining a proximal end and a distal end and a first series of guides extending proximally along the proximal end, any two of the first series of guides defining a gap therebetween, the first series of guides being equally spaced; and
a clamp having a first half section and a second half section, the first half section and the second half section being jointed with a coupling mechanism, each of the first half section and the second half section defining a proximal end and a distal end, each of the first half section and the second half section defining a free end and a fixed end and an arc therebetween, a second series of guides protruding proximally along the proximal end of each leg, any two of the second series of guides defining a gap therebetween.

2. The circumcision device of claim 1, wherein the crown and the clamp are constructed from a biocompatible material.

3. The circumcision device of claim 1, wherein the coupling comprises:
a first boss extending from the first half section, the first boss having a distal protrusion;
a second boss extending from the second half section, the second boss defining an aperture therethrough; and
a cap having a head and a securing protrusion, the securing protrusion defining a central recess, the recess being sized larger than the distal protrusion, the securing protrusion being sized smaller than the internal diameter of the aperture.

4. The circumcision device of claim 3, wherein the coupling mechanism removably attaches the clamp about the crown.

5. The circumcision device of claim 1, wherein the first series of guides about the clamp overlap the second set of guides about the crown when the clamp is properly located about the crown.

6. The circumcision device of claim 1, wherein the clamp further comprises a latching mechanism to secure the first and second half sections in a closed position.

7. A method of circumcising a penis to remove tissue, comprising the steps of:
providing a circumcising device, including:
a crown being oval in shape, the crown defining a proximal end and a distal end and a first series of guides extending proximally along the proximal end, each of the first series of guides defining a first gap therebetween, the first series of guides being equally spaced; and
a clamp having a first half section and a second half section, the first half section and the second half section being joined with a coupling mechanism, each of the first half section and the second half section defining a joined end and a free end and an arc therebetween, a second series of guides extending proximally along the proximal end of each leg, each of the second series of guides defining a second gap therebetween;
placing the crown about the glans penis;
pulling the foreskin over the crown;
holding the foreskin in place over the crown and locating the clamp over the foreskin and about the crown such that each of the second series of guides is aligned with a guide from the first series of guides and each of the second gaps is aligned with one of the first gaps;
securing the clamp about both the foreskin and the crown;
removing captured foreskin by cutting the foreskin with a scalpel along a proximal edge of the guides;
inserting and tying a suture between in the foreskin extending from each of the first and second gaps;
removing the clamp;
removing the crown.
